SEQUENCE LISTING (5 sequences)

SEQ ID NO: 1 - DNA, 214 bp, Artificial Sequence
  miRNA for targeting Drosophila suzukii myd88 gene
tttaaagtccacaactcatcaaggaaaatgaaagtcaaagttggcagcttacttaaacttaatcacagcctttaatgtaaaattaaaaaaaaatagtactataagttaatataccatatctatattactatttttttttaattttgtacctaaagtgcctaacatcattatttaattttttttttttttggcacacgaataaccatgccgtttt

SEQ ID NO: 2 - DNA, 213 bp, Artificial Sequence
  miRNA for targeting Drosophila suzukii myd88 gene
ctttaaagtccacaactcatcaaggaaaatgaaagtcaaagttggcagcttacttaaacttaatcacagcctttaatgttcccgcgcttcatcgttttctttaagttaatataccatatctaaataaaacgatgaagcgcgggagtacctaaagtgcctaacatcattatttaattttttttttttttggcacacgaataaccatgccgtttt

SEQ ID NO: 3 - DNA, 213 bp, Artificial Sequence
  miRNA sequence for targeting Drosophila suzukii myd88 gene
atttaaagtccacaactcatcaaggaaaatgaaagtcaaagttggcagcttacttaaacttaatcacagcctttaatgtacgtcccgttgataaatacctataagttaatataccatatctatatgtatttatcaacgggacgtgtacctaaagtgcctaacatcattatttaattttttttttttttggcacacgaataaccatgccgtttt

SEQ ID NO: 4 - DNA, 2632 bp, Drosophila suzukii
  misc_feature (135)..(135): n is a, c, g, or t
ctgctgaaaccatcggcgtaaacctctaattaaggctagtaacctttgtagaaaattatttagtttatatttttaaacataaattatttttgaaattgtaactaaaaatgtatgcctattttaaaaattccttcntaagaaaattagtttaagtagtacacttttgacgctcactgtatcaaaatttttctggagcgccatctggggagcttactcaatttcaaaagctttactttacttaggtaaagggccgaagataaaagctgacgtaggattttaactgaatgggagctttctaaggttgtttatgcactggagagatagtagataaatgcacttcccacagaacccagagctttcggatctgaaggtcagttgggacttggacccacaagtcgagcttagtttaatagtcagcgcgcttaaacgacgacaactgcacggcgcgcccccattaataatatatatttttccaaaaataaacttaaaaaaaataattaaataaaaaataatattaacattgaacgcgcggtgcacgattttttgacaacaattcagtttcgcttttccttagatttccatataattttttcctttgtgttttccacacacgtggtgttcttctgttactcgcacttcggtagtggttattttttttgggttacattgaaaaaagtacatcgactgccagccgaattcccattcgaaccttaatttgaccaattgacggaaaattaattgcgcacaaaattaattagcaagcgaatatatatttttttttttacgtgaaacgagcgtgttatttgtcaaaaagattttacaactgattgtgtttaagtaaattaacctgagatatatctatgtgtttgtgctacgatccgaaattccaatattgcggtaagtggtgtgatttttggcgggaataaaatcggtttccctttgcgtcgtatttctctcccgatttttctggcgccaaaatgaatgaaaacggttggcaacattttcaacttaacatggcaacatgcgaatctaaccgctgttaggggaacttgtaagggaacatgttagaaattctggaacacctctccaactttgttttgtttggcgctctctctttgaaatgtagaatctcttattttggaacgggagcggttcattgacccagcaacaacaatattcgccatatcttctccttcccagcgatttgcagaattcatccgtactacagttagaaataattctagtccaacagtaagcaaagtcgatcggcgaaatcgggagccgcgttcagttgccttcgagccgtacttagaagtggtagagcccccaggaatcggagagattatcccgtaacttgatactctgacgaaatcccccagacagacccctcgcaatcacacacagctatatactgatatatagatctatatatattgcgcagatcaccctttaaatgcctttcgtccagagattccaacaaatacgtaattttgttatgaacgtctgcctcgtcgaggccatccaattcgtgcaggacctcacgcatgccctctgcctggacctccagggcctgttcaacctcaagtagagattcccgagcgatctcccagcggtaagtcacgccatttggccgcgggccgcgaaaagtgaaagtgcgggagaaattacggttatcgggttttcgggagaaaggggaaagtggaaacctcatggcagcagccctccattttgagaggttatgtcaaaggaaaggaagagaaaggaagttaaaaaatgttagcaaggaaaatattagccttcgtttttcataagtacaccaataaaccacattttactgggtgtcaaaagttagagaattaaacagcaagttaccaaaattagtttgaaggcaaaactttagctagttacgtttttagagggttctggaattacaaatgcccacagcaggagaaatataaatttgtgttttcattttaaacagattatgttttaaaaagtaatacatcaaaattgactttagccgttttttaaataggtaatttggtatataaacatacattttttaataaaaatatgaacactttgtttttatttttgtactggtaggcaaactacaagatataactgttaaataaaaaaagattttaagtgtaggcaataaaaaatcaaatcaaaaatatattttgaataaaagtgaaggtcaaaaatgaaagacttcaaagtaccactctactcatgaagctataataataatgggtaaataaacagtatattacatgttcgttttaaatattttcaatgcaaaaatgccctgcttatattaagctttactttcaccacaactgttcccatcatctatttttaatattattgacctcggacttcttacatttcatcgccagctcatcccatcaatcacccagataaactaccaccttcattaacacaccgcaagcatctcacaacgctgaccaataaacgcctcgacatccagaatccccaactgagacggacgttttgcttggtttttaaaactaaacgaccctttggtagccgcacctattgacctttcacacattcatatccctcggtccacaagccgacccactgatccattcattcacctcccacg

SEQ ID NO: 5 - DNA, 3267 bp, Artificial Sequence
  Antidote transgene sequence including bottleneck promoter, myd88 coding sequence, and SV40 3′ UTR
atgaaccctattaagatcccgacacctcttttcgcctttctgcataataatttcgccattccaccgctgtataattgcgtattcgccttcagtaaatgaaaattgcagttgtattttataatttcgtttttcattttccctctctcattttctacgtctgtttggccagctgtcaattgccgagtgtcgactgaattattggccattaattgcattgattttgattgtaatgaaccagctaatgaaacgtcaacagctaagagtgggccataaaacccggaggaattatcttctagaaaccttgaacatctgtcgcgaatttaagttttaagctgtttatgaattagtaccctcgctaatccttttcgaaaggaccttataaaagtgcgcggattagcaaaaagatttgtgtaaaattggtgcgcggaaaacacgcaaaattcgcgtgctcgggttgcaattaccgggaaatgtgggccttttttacttgcagggcttaggtagtttcccggaatggagtcaagctaagctagccaggtacctgagttccccggaccagctgtccggtgctgaaactagagcaggtagtccccagaatcccagctatataaggcctgccttcctggcaacagcatcacattcgttcatcagccttcaaccctaggatgcgccctcgatttgtatgccatcagcagcactcggtggcccattcccacttccatcccctgtctcacttccagacccattcccatacccatacccacttccagcgccatcccaatccccatccccatccccatcacttttacgacgccactgacgtcggctatcggcgttatcgcaccgccaacatggtggtggccgagggagttatggactccggatcggggatcggtacgggaatgggaatggggcacttcaacgagacccccttatccgagctgggcgtggagacccgctcgcagctgtcccgcatgctgaaccgcaagaaggtgctgcgctccgaggagggctaccagcgggactggcggggcatctcggagctggccaagcagaagggattcgtcgacgagaacgccaacaatcccatggatctggtgctgatcagctggagccagcggagcccgcaaaccgccaaggtgggccatctcgagaacttcctgggcatcatcgatcggtgggatgtctgcgacgatatccaggagaatctggccaaggacacccggcgcttcatcataaagcaggagcagcggcagacctctctggtggaggcgtgtcccccgccccccagcgactgtttcgagaccaacaacaactacagcagcaacaacaacatcacagtgggccaaagtgtccagatcctgagcgacgaggatcagagatgtgtgcagatgggccaaccgctgcccagatacaatgcctgtgtcttgtacgcagaggcagacatcgatcatgccaccgagatcatgaataacctagagtctgagcgatacaatctcaggcttttcctgcgccatcgcgacatgctaatgggcgtacccttcgagcatgtccaactctcccacttcatggccacccgctgtaatcacctgatcgtcgtgctcacagaggagtttcttcggagtccggagaacacgtacctcgtgaacttcacccagaagatacaaatcgagaaccacactcgcaagatcataccgattctgtacaagccagacatgcacataccccagaccctgggcatctatacgcacatcaagtacgccggggactccaagttgttcaacttctgggacaagttggctcgatcgctgcacgatctggatgcctgttccatttactccacgcgccaggtgcaaacaccctcgccagtggaggaatcgactccccagcgggtgaccacgcccagcattcggatacagatcaacgacaaggatgtgaccgacatgcccaacttcaaggtgccggaggcggagaccaccatcgtttcggtatccggcgataccggttcccctctgccggaacacaagccgaagaaaaaggatcgctttctgcgaagaatcacgcacagtttcgccaagacaccgaagaatgaggggagcagtgcgaagaccctgcgacacgcgcactccgtcagcaccataaatgtcacggaacgggagaggacactcagtgccagcagctccaatatctccaccacctcggagagcaagaagagcttcatcaagtggcagccgaatatcctgaagaaggccctattctcccgatccagcaacaagctacagacgccgggttgacatatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatggaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggttccaagcgctccgtacgcgtatcgataagctttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcgg

(19) United States
(12) Patent Application Publication
(10) Pub. No.: US 2020/0270634 A1
(43) Pub. Date: Aug. 27, 2020

(54) USE OF MEDEA ELEMENTS FOR BIOCONTROL OF D. SUZUKII POPULATIONS

(71) Applicant: THE REGENTS OF THE UNIVERSITY OF CALIFORNIA, Oakland, CA (US)

(72) Inventors: Omar S. AKBARI, Riverside, CA (US); Anne BUCHMAN, Riverside, CA (US)

(73) Assignee: The Regents of the University of California, Oakland, CA (US)

(21) Appl. No.: 15/776,281

(22) PCT Filed: Jan. 25, 2017
(86) PCT No.: PCT/US2017/014846

§ 371 Date: May 15, 2018

Related U.S. Application Data
(60) Provisional application No. 62/286,946, filed on Jan. 25, 2016.

(51) Int. Cl. C12N 15/85; C07K 14/435; C12N 15/113; C12N 9/22; A01K 67/033

(52) U.S. Cl. CPC C12N 15/8509; C07K 14/43581; C12N 15/113; A01K 2227/706; A01K 67/0339; C12N 2310/141; A01K 2217/05; C12N 9/22

(57) ABSTRACT

An insect gene drive system for biocontrol of a population of an insect is provided. The gene drive system includes: a) a first DNA sequence encoding a toxin under the control of a maternal germline-specific promoter active in the insect, with the first DNA sequence being linked to b) a second DNA sequence encoding an antidote under the control of an early embryo-specific promoter active in the insect. The toxin is expressed in maternal germline cells of the insect and results in maternal-effect lethality in the insect, and the antidote is expressed in embryos of the insect and counters the maternal-effect lethality. In some embodiments, the insect is Drosophila suzrukii.

### CROSS-REFERENCE TO RELATED APPLICATIONS

This application claims the benefit of Provisional Patent Application No. 62/286,946, filed on Jan. 25, 2016, which is incorporated by reference herein.

### REFERENCE TO SEQUENCE LISTING

A Sequence Listing is submitted electronically via EFS-Web as an ASCII formatted text file with the name “1279611XPCSequenceListing”; the file was created on Jan. 23, 2017, is 9.45 kilobytes in size, and is incorporated herein by reference in its entirety.

### BACKGROUND

### Field of the Invention

The invention relates to a composition and method for a gene drive system.

### Related Art

Spotted wing Drosophila, D. suzukii, is a pest of many small and soft fruits, including cherries, raspberries, blackberries, blueberries, strawberries, peaches, grapes, and others (Walsh et al. 2011). It damages these fruits by using its heavily sclerotized and serrated ovipositor to pierce fruits and lay eggs inside the fruit. Most of the damage caused by D. suzukii is a result of larvae feeding on fruit flesh. However, the insertion of the prominent ovipositor into the skin of the fruit can also cause physical damage to the fruit, as it provides access to secondary infections of pathogens—such as fungi, yeasts and bacteria—that may cause faster fruit deterioration and further losses. These damages can result in severe crop losses, and the implications for exporting producers may also be severe, depending on any quarantine regulations.

A native of eastern and southeastern Asia, D. suzukii has invasively spread in the last several decades, and has been recorded in China, India, Italy, Spain, Russia, and a number of other countries (Walsh et al. 2011). D. suzukii has also rapidly invaded the U.S.—initially found in California in 2008, it has spread to much of the Pacific Coast, to the East Coast, and to north central and interior U.S. (Asplen et al. 2015)—and poses a significant threat to fruit industries there. For example, in 2008, D. suzukii caused over $38.3 million in cherry crop loss in California alone, and is predicted to result in up to $500 million in annual losses in U.S. Western fruit production areas (Goodhue et al., 2011).

Current method of control of D. suzukii rely heavily on the use of expensive non-insect specific insecticides (e.g., malathion), which have variable efficacy (Asplen et al. 2015) and also kill beneficial insects like pollinators (e.g., honeybees) and useful predator (e.g., green lacewings, which prey on various harmful insects like black cherry aphids and small caterpillars). As an alternative to insecticides, farmers can also attempt to trap D. suzukii using chemical attractants; however, no D. suzukii specific attractants are currently available (Walsh et al. 2011), and this approach is not particularly effective at preventing D. suzukii spread. And while biological control of D. suzukii may be possible via use of recently identified natural predators (Gabarra et al. 2015), no established means of D. suzukii biocontrol currently exists (Woltz et al. 2015). Overall, given the rapid spread and potential economic impact of D. suzukii, effective control measures are urgently needed.

### SUMMARY

A Medea system is developed in D. suzukii that can be used as a form of biocontrol. Other similar systems can be built in D. suzukii and in related fly pests, such as in the Caribbean fruit fly, Anastrepha suspense, the Mexican fruit fly, Anastrepha ludens, the West Indian fruit fly, Anastrepha oblique, and other insect pests.

In one aspect, a gene drive system for biocontrol of a Drosophila suzukii population is provided. The gene drive system includes: a) a first DNA sequence encoding a toxin under the control of a maternal germline-specific promoter active in D. suzukii, with the first DNA sequence being linked to b) a second DNA sequence encoding an antidote under the control of an early embryo-specific promoter active in D. suzukii. In this D. suzukii gene drive system, the toxin is expressed in D. suzukii maternal germline cells and results in maternal-effect lethality in D. suzukii, and the antidote is expressed in D. suzukii embryos and counters the maternal-effect lethality.

In another aspect involving the D. suzukii gene drive system, a transgenic D. suzukii whose genome includes the D. suzukii gene drive system is provided.

In a further aspect involving the D. suzukii gene drive system, a method of manipulating a D. suzukii population is provided. The method includes releasing the transgenic D. suzukii into the population in sufficient numbers to spread the gene drive system through the population. In some embodiments, the gene drive system further includes an effector genetic element, which can result in inducible lethality in one or both sexes of D. suzukii, or in recessive sterility in one or both sexes of D. suzukii. The population can be a laboratory or wild population.

More generally, in another aspect, an insect gene drive system for biocontrol of a population of an insect is provided. The gene drive system includes: a) a first DNA sequence encoding a toxin under the control of a maternal germline-specific promoter active in the insect, with the first DNA sequence being linked to b) a second DNA sequence encoding an antidote under the control of an early embryo-specific promoter active in the insect. In this system, the toxin is expressed in maternal germline cells of the insect and results in maternal-effect lethality in the insect, and the antidote is expressed in embryos of the insect and counters the maternal-effect lethality.

In some embodiments involving the insect gene drive system, the insect is not Drosophila melanogaster or a flour beetle. In some embodiments, the insect can be Drosophila suzukii, Anastrepha suspensa, Anastrepha ludens, Anastrepha oblique, Bactrocera oleae/Dacus oleae, Ceratitis capitata, Aedes aegyptii, Anopheles gambiae, or any other insect in which a gene drive system may be of utility.

In a further aspect involving the insect gene drive system, a transgenic insect whose genome includes the insect gene drive system is provided. In some embodiments, the transgenic insect is not Drosophila melanogaster or a flour beetle. In some embodiments, the transgenic insect can be Drosophila suzukii, Anastrepha suspensa, Anastrepha ludens, Anastrepha oblique, Bactrocera oleae/Dacus oleae, Ceratitis capitata, Aedes aegyptii, or Anopheles gambiae.

In another aspect involving the insect gene drive system, a method of manipulating an insect population is provided. The method includes releasing the transgenic insect into a population of the same species in sufficient numbers to spread the gene drive system through the population. In some embodiments, the transgenic insect is not Drosophila melanogaster or a flour beetle. In some embodiments, the transgenic insect can be Drosophila suzukii, Anastrepha suspensa, Anastrepha ludens, Anastrepha oblique, Bactrocera oleae/Dacus oleae, Ceratitis capitata, Aedes aegyptii, or Anopheles gambiae. In some embodiments, the gene drive system further includes an effector genetic element, which can result in inducible lethality in one or both sexes of the insect, or in recessive sterility in one or both sexes of the insect. The population can be a laboratory or wild population.

In any embodiment involving the D. suzukii gene drive system or the more general insect gene drive system:

a) the toxin can include one or more miRNAs, one or more RNA-guided endonucleases, or a combination thereof;

b) the toxin can target a gene for a maternally-deposited embryonic-essential RNA, a maternally-deposited embryonic-essential protein, a zygotically-expressed embryonic essential gene, and the like, of D. suzukii or another insect;

c) the target gene can be the myd88 gene of D. suzukii or another insect;

d) the antidote can be a toxin-resistant version of the target gene, or another gene that can substitute for/fulfill the biological role of the target gene; in some embodiments, the toxin-resistant version is a version not recognized by the toxin;

e) can further include an effector genetic element active in D. suzukii or other insect and linked to the first and second DNA sequences, with the effector genetic element encoding a gene conferring susceptibility to a chemical, a conditional lethal gene, a genetic element that disrupts a recessive fertility gene or recessive lethality gene, or a genetic element that disrupts a gene involved in D. suzukii pest behavior, such as ovipositor formation, olfaction, egg laying substrate preference and other such behaviors; or any other genetic element that reduces or eliminates the effect of D. suzukii pest behavior or the effect of pest behavior of another insect; or

f) any combination of a)-e).

### BRIEF DESCRIPTION OF THE DRAWINGS

For a more complete understanding of the present invention, reference is now made to the following descriptions taken in conjunction with the accompanying drawings, in which:

FIG. 1 is a panel describing Medea genetics. (1A) Heterozygous females carrying Medea cause death of all offspring that fail to inherit Medea. (1B) Synthetic Medea elements contain two genes—maternally expressed miRNAs (the toxin) that silence the expression of a maternally expressed transcript (top line) that normally provides a product essential for early embryonic development, and a zygotic antidote consisting of the silenced maternal mRNA (resistant to the miRNA toxin) sufficient to rescue normal development (vertical line). Adapted from Akbari et al. 2012.

FIG. 2 is a schematic drawing of a portion of a P transposable element vector including, 5′ to 3′, a Hr5Ie1-drived dsRed marker, miRNAs driven by the BicC promoter, recoded Myd88 CDs driven by the bnk promoter, and a 3×P3-GFP marker.

### DETAILED DESCRIPTION

An alternative approach that would complement current control methods would be the use of genetically engineered D. suzukii as a biological control agent. Use of genetically modified insects for wild population manipulation was first suggested many decades ago (Serebrovskii 1940; Hamilton 1967; Curtis 1968), and has garnered a considerable amount of interest since (Burt 2014). Proposed methods typically rely on the use of engineered gene drives based on “selfish” genetic elements (SGEs) that function by forcing inheritance in a non-Mendelian fashion, allowing them to increase in frequency with each generation even without conferring a fitness advantage upon their hosts (Burt 2014; Alphey 2014; Bull 2015). Such methods can be utilized to spread desirable genes linked to the gene drive through a population or to suppress target insect populations (Sinkins and Gould 2006).

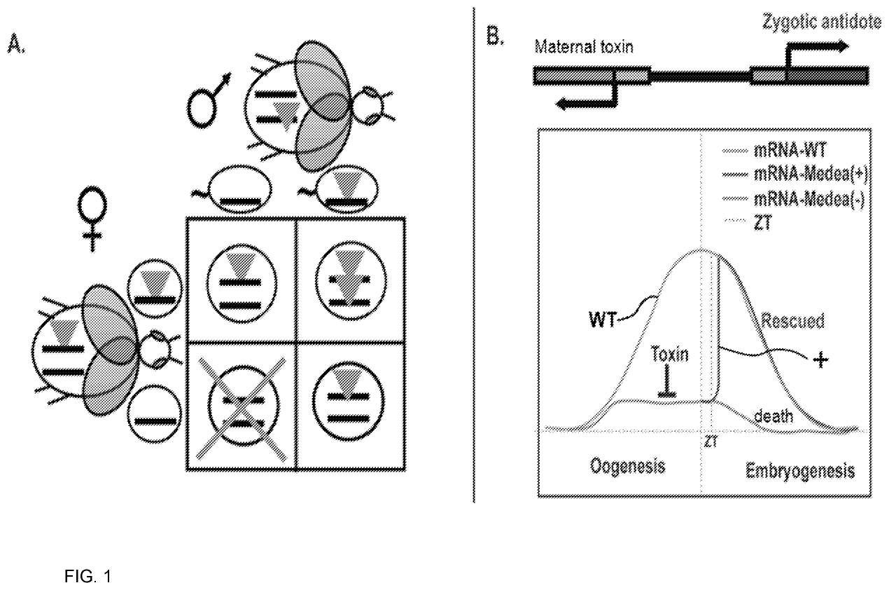

One type of gene drive system is the maternal effect dominant embryonic arrest (Medea). Medea was first discovered in the flour beetle (Wade and Beeman 1994), and multiple versions were later reverse engineered from scratch and shown to act as robust gene drives in the laboratory fruit fly, Drosophila melanogaster (Chen et al. 2007; Akbari et al. 2013). Such engineered Medea systems rely on a Medea element consisting of a toxin-antidote combination. The toxin consists of a miRNA that is expressed during oogenesis in Medea-bearing females, disrupting an embryonic essential gene. A linked antidote is expressed early during embryogenesis and consists of a recoded version of the target gene that is resistant to the miRNA. This combination results in the survival of half of the embryos originating from a Medea-bearing heterozygote female, as those that do not inherit the Medea element die. If a heterozygous Medea female has mated with a heterozygous Medea male, the antidote from the male will also take effect in the embryo, resulting in ¾ of the embryos surviving (FIG. 1). Therefore, Medea will rapidly spread through a population, carrying any linked genes with it.

In the case of D. suzukii, since elimination of the pest population is ultimately of interest, an engineered Medea system could spread a gene proffering susceptibility to a particular pesticide, or a conditional lethal gene that would be activated by some substance or environmental cue such as diapause (a state that allows insects survive periods of adverse conditions such as cold; Clark et al. 2008). For example, a Medea element can be used to spread a gene conferring sensitivity to a particular chemical that is normally innocuous, rendering such a chemical capable of being used as an environmentally-friendly, species-specific pesticide. Trigger-inducible transcription control elements—ones that turn on expression in the presence of a chemical such as tetracycline or vanillic acid (Urlinger et al. 2000; Gitzinger et al. 2012)—can be engineered to drive expression of an insect-specific toxin (e.g., Fu et al. 2007). A Medea element can also be used to spread a gene under the control of a diapause-induced promoter that will splice to produce a toxin in females only, so that, upon the onset of the diapause-inducing environmental cue, all of the females will perish, causing a population crash (Akbari et al. 2013). Further, if a Medea element is inserted into a fertility gene, it could cause a population crash by spreading through a population and making it infertile as it does. However, although transgenesis of D. suzukii has been established (Schetelig et al. 2013), no gene drive systems in this major pest have yet been engineered.

In embodiments of the D. suzukii gene drive system or the more general insect gene drive system described herein, a toxin can be an miRNA or an RNA-guided endonuclease.

MicroRNAs (miRNAs) mediate the RNAi pathway. The term “microRNA” or “miRNA” as used herein indicates a class of short RNA molecules of about 22 nucleotides in length, which are found in most eukaryotic cells. miRNAs are generally known as post-transcriptional regulators that bind to complementary sequences on target mRNA transcripts, usually resulting in translational repression and gene silencing. miRNAs are encoded by miRNA genes and are initially transcribed into primary miRNAs (pri-miRNA), which can be hundreds or thousands of nucleotides in length and contain from one to six miRNA precursors in hairpin loop structures. These hairpin loop structures are composed of about 70 nucleotides each, and can be further processed to become precursor-miRNAs (pre-miRNA) having a hairpin-loop structure and a two-base overhang at its 3′ end. In the cytoplasm, the pre-miRNA hairpin is cleaved by the RNase III enzyme Dicer. Dicer interacts with the 3′ end of the hairpin and cuts away the loop joining the 3′ and 5′ arms, yielding an imperfect miRNA:miRNA duplex about 22 nucleotides in length. Overall hairpin length and loop size influence the efficiency of Dicer processing, and the imperfect nature of the miRNA:miRNA base pairing also affects cleavage. Although either strand of the duplex can potentially act as a functional miRNA, only one strand is usually incorporated into the RNA-induced silencing complex RISC where the miRNA and its mRNA target interact.

An RNA-guided endonuclease is a nuclease such as CRISPR-associated 9 (Cas9) or Cpf1, that is directed by guide RNAs to target and cleave specific nucleotide sequences either in DNA or RNA. Such an endonuclease may be used to cleave or silence a target gene RNA similarly to an miRNA. Examples of RNA-guided endonucleases include, but are not limited to, Cas9, C2C2, Cpf1, Cas13a, Cas13b, and any other suitable Cas-type proteins.

Other toxins include siRNAs and maternally supplied protein toxins (e.g. cell death genes, restriction endonucleases, insect toxins, e.g., barnase).

The particular toxin that is used will depend on the selected target. A toxin can be prepared by standard molecular biological methods.

Examples of maternally-deposited embryonic-essential RNAs or proteins for use as toxin targets include, but are not limited to, myd88 (NCBI Gene database, Gene ID: 35956), Groucho (NCBI Gene database, Gene ID: 43162), DAH (NCBI Gene database, Gene ID: 32459), O-futl (NCBI Gene database, Gene ID: 36564), homologs of such promoters in other insects, and other D. suzukii and insect promoters identified by analyzing expression data. In some embodiments of the D. suzukii gene drive system or the general insect gene drive system, the toxin target is not myd88 but is another target.

The antidote can be a modified version of a target. The modified version can include nucleotide sequence changes that prevent an miRNA or RNA-guided endonuclease toxin from binding to the modified target sequence. The particular antidote can be prepared by standard molecular biological methods and will depend on the selected target. Examples of other types of antidotes include, but are not limited to, zygotically expressed RNAi, CRISPR, or siRNA to the toxin when the toxin is a protein.

Examples of maternal germline-specific promoters include, but are not limited to, bicoid, vasa, deadhead, zpg, and exu promoters, homologs of such promoters in insects, and promoters of other genes expressed specifically in the female germline.

Examples of early embryo-specific promoters include, but are not limited to, bottleneck, malpha, twi, ocho, and tin promoters, homologs of such promoters in insects, and promoters of other genes expressed specifically in the early embryo.

For a D. suzukii gene drive system or general insect gene drive system that contains an effector genetic element, the components of the system can be organized in various arrangements depending on the nature of the effector element. For example, the effector element can be located downstream of, or between, the first and second DNA sequences, or the first and second DNA sequences can be located within the effector genetic element. Other arrangements are possible as long as the first and second DNA sequences and the effector genetic element sequences are present.

The D. suzukii gene drive system or general insect gene drive system can be present, for example, in a DNA construct, in a nucleic acid vector such as a cloning vector or a P transposable element vector, or in vivo in an insect.

Although D. melanogaster and D. suzukii are closely related, in attempting to transfer the technology described here from the former to the latter, there was no reasonable expectation of success in preparing a D. suzukii gene drive system, and the results described here are unexpected. This is due to two primary reasons.

Firstly, although D. suzukii is a significant threat to agricultural output on a global level (Asplen et al. 2015), it is rather poorly studied compared to the extremely well-understood D. melanogaster, and few genetic tools that allow basic transgene construction and precise genome manipulation have been developed. A draft genome was assembled in 2013 (Chiu et al. 2013), and since then, transgenesis has been demonstrated once (Schetelig and Handler 2013) and a single study has shown that direct injection of CRISPR/Cas9 can result in mutation (Li and Scott 2015). This represents a very small genetic toolkit, especially when compared to other insects where gene drive is of interest, such as mosquitoes (e.g., Esvelt et al. 2014). D. suzukii can be more difficult to work with in the laboratory than D. melanogaster as it has different temperature and other environmental needs (Kinjo et al. 2014), making fewer researchers willing to work with it. The lack of genetic tools and the potential difficulties in rearing the insect possibly explain why no transgenic gene drive components, let alone functional gene drive systems, have yet been created in D. suzukii, despite the need for such elements.

Additionally, the Medea gene system itself is quite difficult to engineer in any organism, especially in one lacking a complete genetic tool kit, when compared to other gene drive systems. As outlined, for example, in Champer et al. (2016), Medea systems have not yet been developed in mosquitoes or other insects besides D. melanogaster, despite researcher efforts. This is due, in part, because a Medea system such as the one demonstrated by Chen et al. (2007) requires effective RNAi-mediated silencing in the germ line, which has been difficult to achieve in species other than D. melanogaster, and also because such a system depends on identification and functional characterization of maternal and zygotic promoters, embryonic essential target genes, and other genetic elements, that is lacking in many target species (including D. suzukii). Simply put, the type of Medea system developed in Drosophila melanogaster is not highly portable to different organisms.

Therefore, the success of such a system in a pest species is unexpected given the knowledge regarding said system in the field of gene drive.

Having demonstrated the transfer of the Medea system to D. suzukii, however, the transfer of the system to other insects is now expected to be successful because the requisite tools (germline RNAi, RNA CRIPSR, etc.) can now be made available, and the proof of principle for D. suzukii described herein shows that attempting this in other insects with similar conserved genes is now feasible.

The present invention may be better understood by referring to the accompanying examples, which are intended for illustration purposes only and should not in any sense be construed as limiting the scope of the invention.

### Example 1

### Methods

Generation and Testing of D. suzukii Medea

To create a Medea-like maternal-effect selfish genetic element in D. suzukii, synthetic Medea elements were engineered based on the same architecture used to generate the Medead^{myd88} system previously built in D. melanogaster (Chen et al. 2007). In D. melanogaster, maternal Myd88 is required for dorsal-ventral patterning in early embryo development, and germ-line loss-of-function myd88 mutant females produce embryos that fail to hatch (Kambris et al. 2003). Myd88 is highly conserved in D. suzukii (and in many other Drosophilids), and it was reasoned that Myd88 would likely be essential in this species, as well.

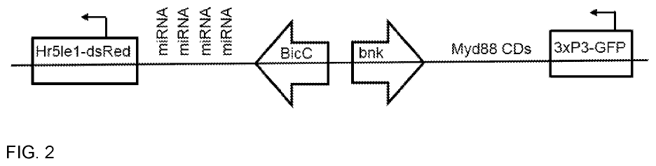

Briefly, a P transposable element vector was generated in which the predicted D. suzukii female germ-line-specific bicoid (bic) promoter drives the expression of a “toxin” containing three synthetic microRNAs (miRNAs) designed to target the 5′ untranslated region (UTR) of D. suzukii myd88. (The synthetic miRNAs were generated using the mir6.1 backbone, as described by Chen et al. 2007.) The vector was generated by cloning DNA pieces generated by PCR into a standard Drosophila melanogaster P element vector via Gibson assembly (Gibson D G, Young L, Chuang R Y, Venter J C, Hutchison C A 3rd, Smith H O (2009). “Enzymatic assembly of DNA molecules up to several hundred kilobases”. Nature Methods. 6 (5): 343-345). The vector also contains an “antidote” transgene containing D. suzukii myd88, recoded to be insensitive to the miRNAs, expressed under control of the early embryo-specific bottleneck (bnk) promoter (Schejter et al. 1993). The vector also contained two separate transformation markers—GFP under control of the eye-specific 3×P3 promoter (Berghammer et al. 1999), and dsRed under control of the ubiquitous opie2 promoter (Theilmann and Stewart, 1992) (FIG. 2).

The sequences of the miRNAs used are as follows, with guide and passenger strands capitalized:

a) miRNA 1:
(SEQ ID NO. 1)
tttaaagtccacaactcatcaaggaaaatgaaagtcaaagttggcagctt
acttaaacttaatcacagcctttaatgtAAAATTAAAAAAAAATAGTACT
AtaagttaatataccatatctaTATTACTATTTTTTTTTAATTTTgtacc
taaagtgcctaacatcattatttaattttttttttttttggcacacgaat
aaccatgccgtttt
b) miRNA 2:
(SEQ ID NO. 2)
ctttaaagtccacaactcatcaaggaaaatgaaagtcaaagttggcagct
tacttaaacttaatcacagcctttaatgtTCCCGCGCTTCATCGTTTTCT
TtaagttaatataccatatctaAATAAAACGATGAAGCGCGGGAgtacct
aaagtgcaaacatcattatttaattttttttttttttggcacacgaataa
ccatgccgtttt
c) miRNA 3:
(SEQ ID NO. 3)
atttaaagtccacaactcatcaaggaaaatgaaagtcaaagttggcagct
tacttaaacttaatcacagcctttaatgtACGTCCCGTTGATAAATACCT
AtaagttaatataccatatctaTATGTATTTATCAACGGGACGTgtacct
aaagtgcaaacatcattatttaattttttttttttttggcacacgaataa
ccatgccgtttt

The sequence of the miRNA-expressing promoter (predicted bicoid promoter) used is as follows:

(SEQ ID NO. 4)
CTGCTGAAACCATCGGCGTAAACCTCTAATTAAGGCTAGTAACCTTTGTA
GAAAATTATTTAGTTTATATTTTTAAACATAAATTATTTTTGAAATTGTA
ACTAAAAATGTATGCCTATTTTAAAAATTCCTTCNTAAGAAAATTAGTTT
AAGTAGTACACTTTTGACGCTCACTGTATCAAAATTTTTCTGGAGCGCCA
TCTGGGGAGCTTACTCAATTTCAAAAGCTTTACTTTACTTAGGTAAAGGG
CCGAAGATAAAAGCTGACGTAGGATTTTAACTGAATGGGAGCTTTCTAAG
GTTGTTTATGCACTGGAGAGATAGTAGATAAATGCACTTCCCACAGAACC
CAGAGCTTTCGGATCTGAAGGTCAGTTGGGACTTGGACCCACAAGTCGAG
CTTAGTTTAATAGTCAGCGCGCTTAAACGACGACAACTGCACGGCGCGCC
CCCATTAATAATATATATTTTTCCAAAAATAAACTTAAAAAAAATAATTA
AATAAAAAATAATATTAACATTGAACGCGCGGTGCACGATTTTTTGACAA
CAATTCAGTTTCGCTTTTCCTTAGATTTCCATATAATTTTTTCCTTTGTG
TTTTCCACACACGTGGTGTTCTTCTGTTACTCGCACTTCGGTAGTGGTTA
TTTTTTTTGGGTTACATTGAAAAAAGTACATCGACTGCCAGCCGAATTCC
CATTCGAACCTTAATTTGACCAATTGACGGAAAATTAATTGCGCACAAAA
TTAATTAGCAAGCGAATATATATTTTTTTTTTTACGTGAAACGAGCGTGT
TATTTGTCAAAAAGATTTTACAACTGATTGTGTTTAAGTAAATTAACCTG
AGATATATCTATGTGTTTGTGCTACGATCCGAAATTCCAATATTGCGGTA
AGTGGTGTGATTTTTGGCGGGAATAAAATCGGTTTCCCTTTGCGTCGTAT
TTCTCTCCCGATTTTTCTGGCGCCAAAATGAATGAAAACGGTTGGCAACA
TTTTCAACTTAACATGGCAACATGCGAATCTAACCGCTGTTAGGGGAACT
TGTAAGGGAACATGTTAGAAATTCTGGAACACCTCTCCAACTTTGTTTTG
TTTGGCGCTCTCTCTTTGAAATGTAGAATCTCTTATTTTGGAACGGGAGC
GGTTCATTGACCCAGCAACAACAATATTCGCCATATCTTCTCCTTCCCAG
CGATTTGCAGAATTCATCCGTACTACAGTTAGAAATAATTCTAGTCCAAC
AGTAAGCAAAGTCGATCGGCGAAATCGGGAGCCGCGTTCAGTTGCCTTCG
AGCCGTACTTAGAAGTGGTAGAGCCCCCAGGAATCGGAGAGATTATCCCG
TAACTTGATACTCTGACGAAATCCCCCAGACAGACCCCTCGCAATCACAC
ACAGCTATATACTGATATATAGATCTATATATATTGCGCAGATCACCCTT
TAAATGCCTTTCGTCCAGAGATTCCAACAAATACGTAATTTTGTTATGAA
CGTCTGCCTCGTCGAGGCCATCCAATTCGTGCAGGACCTCACGCATGCCC
TCTGCCTGGACCTCCAGGGCCTGTTCAACCTCAAGTAGAGATTCCCGAGC
GATCTCCCAGCGGTAAGTCACGCCATTTGGCCGCGGGCCGCGAAAAGTGA
AAGTGCGGGAGAAATTACGGTTATCGGGTTTTCGGGAGAAAGGGGAAAGT
GGAAACCTCATGGCAGCAGCCCTCCATTTTGAGAGGTTATGTCAAAGGAA
AGGAAGAGAAAGGAAGTTAAAAAATGTTAGCAAGGAAAATATTAGCCTTC
GTTTTTCATAAGTACACCAATAAACCACATTTTACTGGGTGTCAAAAGTT
AGAGAATTAAACAGCAAGTTACCAAAATTAGTTTGAAGGCAAAACTTTAG
CTAGTTACGTTTTTAGAGGGTTCTGGAATTACAAATGCCCACAGCAGGAG
AAATATAAATTTGTGTTTTCATTTTAAACAGATTATGTTTTAAAAAGTAA
TACATCAAAATTGACTTTAGCCGTTTTTTAAATAGGTAATTTGGTATATA
AACATACATTTTTTAATAAAAATATGAACACTTTGTTTTTATTTTTGTAC
TGGTAGGCAAACTACAAGATATAACTGTTAAATAAAAAAAGATTTTAAGT
GTAGGCAATAAAAAATCAAATCAAAAATATATTTTGAATAAAAGTGAAGG
TCAAAAATGAAAGACTTCAAAGTACCACTCTACTCATGAAGCTATAATAA
TAATGGGTAAATAAACAGTATATTACATGTTCGTTTTAAATATTTTCAAT
GCAAAAATGCCCTGCTTATATTAAGCTTTACTTTCACCACAACTGTTCCC
ATCATCTATTTTTAATATTATTGACCTCGGACTTCTTACATTTCATCGCC
AGCTCATCCCATCAATCACCCAGATAAACTACCACCTTCATTAACACACC
GCAAGCATCTCACAACGCTGACCAATAAACGCCTCGACATCCAGAATCCC
CAACTGAGACGGACGTTTTGCTTGGTTTTTAAAACTAAACGACCCTTTGG
TAGCCGCACCTATTGACCTTTCACACATTCATATCCCTCGGTCCACAAGC
CGACCCACTGATCCATTCATTCACCTCCCACG

The antidote transgene sequence used, including the bottleneck promoter, MYD88 Coding Sequence, and SV4O 3′ UTR, is as follows (MYD88 coding region capitalized):

(SEQ ID NO. 5)
atgaaccctattaagatcccgacacctcttttcgcctttctgcataataa
tttcgccattccaccgctgtataattgcgtattcgccttcagtaaatgaa
aattgcagttgtattttataatttcgtttttcattttccctctctcattt
tctacgtctgtttggccagctgtcaattgccgagtgtcgactgaattatt
ggccattaattgcattgattttgattgtaatgaaccagctaatgaaacgt
caacagctaagagtgggccatanacccggaggaattatcttctagaaacc
ttgaacatctgtcgcgaatttaagttttaagctgtttatgaattagtacc
ctcgctaatccttttcgaaaggaccttataaaagtgcgcggattagcaaa
aagatttgtgtaaaattggtgcgcggaaaacacgcaaaattcgcgtgctc
gggttgcaattaccgggaaatgtgggccttttttacttgcagggcttagg
tagtttcccggaatggagtcaagctaagctagccaggtacctgagttccc
cggaccagctgtccggtgctgaaactagagcaggtagtccccagaatccc
agctatataaggcctgccttcctggcaacagcatcacattcgttcatcag
ccttcaaccctaggATGCGCCCTCGATTTGTATGCCATCAGCAGCACTCG
GTGGCCCATTCCCACTTCCATCCCCTGTCTCACTTCCAGACCCATTCCCA
TACCCATACCCACTTCCAGCGCCATCCCAATCCCCATCCCCATCCCCATC
ACTTTTACGACGCCACTGACGTCGGCTATCGGCGTTATCGCACCGCCAAC
ATGGTGGTGGCCGAGGGAGTTATGGACTCCGGATCGGGGATCGGTACGGG
AATGGGAATGGGGCACTTCAACGAGACCCCCTTATCCGAGCTGGGCGTGG
AGACCCGCTCGCAGCTGTCCCGCATGCTGAACCGCAAGAAGGTGCTGCGC
TCCGAGGAGGGCTACCAGCGGGACTGGCGGGGCATCTCGGAGCTGGCCAA
GCAGAAGGGATTCGTCGACGAGAACGCCAACAATCCCATGGATCTGGTGC
TGATCAGCTGGAGCCAGCGGAGCCCGCAAACCGCCAAGGTGGGCCATCTC
GAGAACTTCCTGGGCATCATCGATCGGTGGGATGTCTGCGACGATATCCA
GGAGAATCTGGCCAAGGACACCCGGCGCTTCATCATAAAGCAGGAGCAGC
GGCAGACCTCTCTGGTGGAGGCGTGICCCCCGCCCCCCAGCGACTGTTTC
GAGACCAACAACAACTACAGCAGCAACAACAACATCACAGTGGGCCAAAG
TGTCCAGATCCTGAGCGACGAGGATCAGAGATGTGTGCAGATGGGCCAAC
CGCTGCCCAGATACAATGCCTGTGTCTTGTACGCAGAGGCAGACATCGAT
CATGCCACCGAGATCATGAATAACCTAGAGTCTGAGCGATACAATCTCAG
GCTTTTCCTGCGCCATCGCGACATGCTAATGGGCGTACCCTTCGAGCATG
TCCAACTCTCCCACTTCATGGCCACCCGCTGTAATCACCTGATCGTCGTG
CTCACAGAGGAGTTTCTTCGGAGTCCGGAGAACACGTACCTCGTGAACTT
CACCCAGAAGATACAAATCGAGAACCACACTCGCAAGATCATACCGATTC
TGTACAAGCCAGACATGCACATACCCCAGACCCTGGGCATCTATACGCAC
ATCAAGTACGCCGGGGACTCCAAGTTGTTCAACTTCTGGGACAAGTTGGC
TCGATCGCTGCACGATCTGGATGCCTGTTCCATTTACTCCACGCGCCAGG
TGCAAACACCCTCGCCAGTGGAGGAATCGACTCCCCAGCGGGTGACCACG
CCCAGCATTCGGATACAGATCAACGACAAGGATGTGACCGACATGCCCAA
CTTCAAGGTGCCGGAGGCGGAGACCACCATCGTTTCGGTATCCGGCGATA
CCGGTTCCCCTCTGCCGGAACACAAGCCGAAGAAAAAGGATCGCTTTCTG
CGAAGAATCACGCACAGTTTCGCCAAGACACCGAAGAATGAGGGGAGCAG
TGCGAAGACCCTGCGACACGCGCACTCCGTCAGCACCATAAATGTCACGG
AACGGGAGAGGACACTCAGTGCCAGCAGCTCCAATATCTCCACCACCTCG
GAGAGCAAGAAGAGCTTCATCAAGTGGCAGCCGAATATCCTGAAGAAGGC
CCTATTCTCCCGATCCAGCAACAAGCTACAGACGCCGGGTTGAcatatgg
atctttgtgaaggaaccttacttctgtggtgtgacataattggacaaact
acctacagagatttaaagctctaaggtaaatataaaatattaagtgtata
atgtgttaaactactgattctaattgtttgtgtattttagattccaacct
atggaactgatgaatgggagcagtggtggaatgcctttaatgaggaaaac
ctgttttgctcagaagaaatgccatctagtgatgatgaggctactgctga
ctctcaacattctactcctccaaaaaagaagagaaaggtagaagacccca
aggactttccttcagaattgctaagttttttgagtcatgctgtgtttagt
aatagaactcttgcttgctttgctatttacaccacaaaggaaaaagctgc
actgctatacaagaaaattaggaaaaatatttgatgtatagtgccttgac
tagagatcataatcagccataccacatttgtagaggttttacttgcttta
aaaaacctcccacacctccccctgaacctgaaacataaaatgaatggaat
tgttgttgttaacttgtttattgcagcttataatggttacaaataaagca
atagcatcacaaatttcacaaataaagcatttttttcactgcattctagt
tgtggtttgtccaaactcatcaatgtatcttatcatgtctggttccaagc
gctccgtacgcgtatcgataagctttaagatacattgatgagtttggaca
aaccacaactagaatgcagtgaaaaaaatgctttatttgtgaaatttgtg
atgctattgctttatttgtaaccattataagagcaataaacaagttaaca
acaacaattgcattcattttatgtttcaggttcagggggaggtgtgggag
gttttttaaagcaagtaaaacactacaaatgtggtatgactgattatgat
ctagagtcgcgg

The Genbank accession number for the D. suzukii MYD88 sequence is GenBank: KI419649.1 (incorporated by reference herein).

The Accession number for the D. suzukii genome sequence is Dsuzukii.v01 [GCA_000472105.1] (incorporated by reference herein).

The vector, along with a source of transposase, was injected into D. suzukii embryos using standard injection procedures as described in The Use of P-Element Transposons to Generate Transgenic Flies (Bachmann and Kust, Chapter 4) from Methods in Molecular Biology: Drosophila: Methods and Protocols Edited by: C. Dahmann C Humana Press Inc., Totowa, N.J., and the surviving G0 adults were individually outcrossed to wild type (WT) individuals. G1 progeny were screened for the presence of the Medea element (as evidenced by ubiquitous dsRed expression), and one G1 transformant male was recovered. When outcrossed to WT virgin females, the male produced 50% Medea+ and 50% WT individuals (consistent with Mendelian transmission ratios). The Medea+G2 progeny were further individually outcrossed to WT individuals of the opposite sex. From these crosses, the males (n=3) gave rise to ˜50% Medea+ progeny; the females (n=9) gave rise to 100%0/Medea+ progeny, which is consistent with the Medea element functioning as a maternal effect dominant embryonic arrest system. Of the G3 Medea+ progeny, nine males and 32 virgin females were further individually outcrossed to WT. The males have rise to 50% Medea+:50% WT individuals, and male outcrosses were discontinued at this stage. The females all gave rise to 100%/Medea+ offspring (with a mean=9.34 G4 progeny recovered).

Fifty G4 heterozygous Medea+ virgin females were further individually outcrossed to WT males, and all of their progeny (n=603) were Medea+. Thirty-one resulting G5 heterozygous Medea+ virgin females were then outcrossed further. In this outcross, a small number of progeny that were negative for the Medea element were recovered for the first time, indicating that the system did not function at 100%. Of the 31 G4 crosses that gave rise to scorable progeny, eight G4 females produced a small number of Medea-offspring (ranging from one to seven per female), while 23 gave rise to 100% Medea+ progeny. Although a Medea system that works perfectly would be ideal, one that gives rise to mostly Medea+ progeny would still be expected to spread through a population. In this case, of the total G5 progeny, almost 97% (n=785) were Medea+, demonstrating that the system functions very efficiently, if not ideally.

Overall, when all generations were summed together, the percentage of Medea+ progeny arising from single heterozygous female outcrosses was nearly 99, with 1788 Medea+ progeny out of 1813 total (summarized in Table 1).

TABLE 1

| Generation | Sex (# crossed out) | Number of Progeny | % Medea |
|---|---|---|---|
| G2 | Female (9) | 126 | 100% |
| | Male (3) | 45 | 50% |
| G3 | Female (32) | 299 | 100% |
| | Male (9) | 130 | 50% |
| G4 | Female (50) | 603 | 100% |
| G5 | Female (31) | 785 | 97% |

| | | |
|---|---|---|
| Total Medea+ from females | 1788/1813 | 99% |

To assess whether the Medea system would function well in geographically distinct populations (which likely harbor genetic variability, especially in regions of the genome that canonically have less conservation, such as the 5′UTR regions), heterozygous Medea+ flies were individually introgressed with nine geographically distinct D. suzukii populations, and resulting Medea+ heterozygous virgins were outcrossed to males from the nine populations to determine whether the Medea element functioned appropriately. Although the rate of inheritance was not 100% (as would be expected if the Medea element functioned perfectly), it was an average of 96%, which is more than sufficient to bring about gene drive (data summarized in Table 2 below). Therefore, the developed Medea system is a gene drive tool for functioning globally, in diverse populations of the D. suzukii pest.

TABLE 2

| Strain origin | # of progeny | % Medea+ |
|---|---|---|
| Clayton, WA | 152 | 88% |
| Watsonville, CA | 62 | 100% |
| Brentwood, CA | 251 | 94% |
| Enime, Japan | 133 | 100% |
| Oahu, HI | 167 | 99% |
| Beltsville, MD | 100 | 98% |
| Oregon | 155 | 100% |
| Tracy, CA | 283 | 88% |
| Maryland | 16 | 100% |
| Total | 1319 | 96% |

### Population Cage Experiments

To determine whether the generated D. suzukii Medea is capable of spreading through populations, population cage experiments were set up as follows. Heterozygous Medea (Medea/+) males and WT (+/+) males were allowed to mate with WT (+/+) females in proportions of 25 Medea/+ males: 25+/+ males: 50+/+ females (for an allele frequency of ˜12.5%) and 30 Medea/+ males: 20+/+ males: 50+/+ females (for an allele frequency of ˜15%). Additionally, heterozygous Medea (Medea/+) males were allowed to mate with WT (+/+) females in proportions of 50 Medea/+ males: 50+/+ females (for an allele frequency of ˜25%), and homozygous Medea males (Medea/Medea) were allowed to mate with WT (+/+) females in proportions of 50 Medea/Medea males: 50+/+ females (for an allele frequency of ˜50%). The total number of flies for each starting population was 100. After being placed together, adult flies were removed after exactly seven days. After another seven days, progeny were collected and separated in half arbitrarily. One half was counted, while the other half was placed in a new bottle to continue the simulation, and this process continued throughout the duration of the experiment. All experiments were conducted in triplicate. All fly experiments were carried out at −20° C. with ambient humidity in 250 ml bottles containing a fly medium prepared based on a recipe from USDS.

These experiments are ongoing; however, when released at a higher threshold, the D. suzukii Medea system does appear to demonstrate gene drive (drive experiments continuing for several more generations should confirm these results). Given the observed genetic behavior of the present Medea system, it is anticipated that the Medea element will spread through the experimental populations in the predicted manner given sufficient time.

### Theoretical Framework

Previous theoretical analyses have shown that Medea elements can spread when introduced into the population at allele frequencies from 2.5% to 25%, depending on the fitness cost (Ward et al. 2011; Marshall and Hay 2013), and population cage experiments in D. melanogaster have practically shown Medea fixation with release frequencies of 25% (Chen et al. 2007; Akbari et al. 2012). These numbers, while high, are relatively small compared with those associated with classical sterile male release in other insects, e.g., up to ˜10′ in the case of Mediterranean fruit flies (Dyck et al. 2005).

Mathematical analyses have also shown that Medea elements can be used for purposes of population suppression. For example, Akbari et al. (2012) have demonstrated that a Medea system comprising a lethality cassette that kills females upon some environmental cue can be used to bring about population suppression by causing a cue-induced population crash.

Preliminary modeling as described herein has also indicated that a Medea element can induce a population crash when inserted into a male or female recessive fertility gene. In this scenario, heterozygous Medea-bearing individuals of one sex would be fertile, while homozygous individuals of that sex would be recessively sterile. Numerous male and female recessive fertility genes have been identified in D. melanogaster and are likely to be conserved in D. suzukii. A Medea element could be inserted into such a gene to disrupt it using a number of techniques, e.g., CRISPR-Cas9 homology directed repair (Mali et al. 2013). In this way, the Medea element would be inexorably linked to the mutation in the fertility gene, and individuals possessing two copies of the Medea element would necessarily be sterile.

### DISCUSSION

In brief, the engineered D. suzukii Medea element discussed here (and other similar elements) allow for manipulation of wild D. suzukii populations in a number of ways. Chiefly, such Medea elements enable the construction of a D. suzukii strain that could be released into the wild to suppress/eradicate the wild population of D. suzukii, by, for example, eliminating the production of females or rendering females or males sterile. A primary appeal of such an approach is that it is 100% insect specific, with only D. suzukii being targeted. Another benefit of this approach is that it is catalytic: modest numbers of engineered insects would need to be released into the wild population for the relevant transgenes to spread into the population and cause a population crash. An important consequence of the fact that the system relies on the engineered insects to do the work of suppression generation after generation is that it is cheap, with only a few releases resulting in suppression of the species on an ongoing basis, as compared to the use of insecticides, which need to be applied on a regular basis.

Although the system described here is based on the Myd88 gene, other Medea systems targeting a variety of genes can be constructed (e.g., Akbari et al. 2012), and should function in the same manner. Second-generation Medea systems that can counteract the population suppression effects of the first system can also be generated, as discussed elsewhere (Chen et al. 2007; Akbari et al. 2012). Additionally, Medea systems can be developed using an RNA-guided endonuclease (instead of miRNAs) as the toxin designed to maternally target the mRNA of a maternally deposited embryonic essential gene, preferably in multiple places to reduce the generation of resistance alleles. And, Medea elements can also be engineered in other species of interest. For example, a Medea system of the kind described here can be generated in a number of fly pests related to Drosophila, such as in the Caribbean fruit fly, Anastrepha suspensa, the Mexican fruit fly, Anastrepha ludens, the West Indian fruit fly, Anastrepha oblique, the olive fruit fly, Bactrocera oleae/Dacus oleae, the Mediterranean fruit fly, Ceratitis capitata, and other insect pests.

### REFERENCES

The following publications are incorporated by reference herein in their entirety:
1. Akbari, O. S., Bellen, H. J., Bier, E., Bullock, S. L., Burt, A., Church, G. M., Cook, K. R., Duchek, P., Edwards, O. R., Esvelt, K. M., et al. (2015). BIOSAFETY. Safeguarding gene drive experiments in the laboratory. Science 349, 927-929.
2. Akbari, O. S., Chen, C. H., Marshall, J. M., Huang, H., Antoshechkin, I., and Hay, B. A. (2014). Novel synthetic Medea selfish genetic elements drive population replacement in Drosophila; a theoretical exploration of Medea-dependent population suppression. ACS synthetic biology 3, 915-928.
3. Akbari, O. S., Matzen, K. D., Marshall, J. M., Huang, H., Ward, C. M., and Hay, B. A. (2013). A synthetic gene drive system for local, reversible modification and suppression of insect populations. Current biology: CB 23, 671-677.
4. Alphey, L. Genetic control of mosquitoes. Annu Rev Entomol 59, 205-24 (2014).
5. Alphey, L. et al. Genetic control of Aedes mosquitoes. Pathog Glob Health 107, 170-9 (2013).
6. Altrock, P. M., Traulsen, A., and Reed, F. A. (2011). Stability properties of underdominance in finite subdivided populations. PLoS computational biology 7, e1002260.
7. Altrock, P. M., Traulsen, A., Reeves, R. G., and Reed, F. A. (2010). Using underdominance to bi-stably transform local populations. Journal of theoretical biology 267, 62-75.
8. Asman, S. M., McDonald, P. T., and Prout, T. (1981). Field studies of genetic control systems for mosquitoes. Annual review of entomology 26, 289-318.
9. Baker, R. H. (1984). Chromosome Rearrangements in the Control of Mosquitos. Prev Vet Med 2, 529-540.
10. Beumer, K. J., Pimpinelli, S., and Golic, K. G. (1998). Induced chromosomal exchange directs the segregation of recombinant chromatids in mitosis of Drosophila. Genetics 150, 173-188.
11. Bull, J. J. (2015). Evolutionary Decay and the Prospects for Long Term Disease Intervention using Engineered Insect Vectors. Evolution, Medicine, and Public Health 1, 152-166.
12. Burt, A. Heritable strategies for controlling insect vectors of disease. Philos Trans R Soc Lond B Biol Sci 369, 20130432 (2014).
13. Burt, A. Site-specific selfish genes as tools for the control and genetic engineering of natural populations. Proc Biol Sci 270, 921-8 (2003).
14. Carroll, D. Genome engineering with targetable nucleases. Annu Rev Biochem 83, 409-39 (2014).
15. Caraballo, H. & King, K. Emergency department management of mosquito-borne illness: malaria, dengue, and West Nile virus. Emerg Med Pract 16, 1-23; quiz 23-4 (2014).
16. Champer, Jackson; Buchman, Anna; Akbari, Omar S. Cheating evolution: engineering gene drives to manipulate the fate of wild populations. Nature Reviews Genetics. 17 (3): 146-159. doi:10. 1038/nrg.2015.34.
17. Chen, C. H., Huang, H., Ward, C. M., Su, J. T., Schaeffer, L. V., Guo, M., and Hay, B. A. (2007). A synthetic maternal-effect selfish genetic element drives population replacement in Drosophila. Science 316, 597-600.
18. Chiu J C, Jiang X T, Zhao L et al (2013) Genome of Drosophila suzukii, the Spotted Wing Drosophila. G3-Genes Genomes Genet 3:2257-2271. doi:10.1534/g3.113.008185
19. Curtis, C. F. (1968). Possible use of translocations to fix desirable genes in insect pest populations. Nature 218, 368-369.
20. Davis, S., Bax, N., and Grewe, P. (2001). Engineered underdominance allows efficient and economical introgression of traits into pest populations. Journal of theoretical biology 212, 83-98.
21. de La Rocque, S., Balenghien, T., Halos, L., Dietze, K., Claes, F., Ferrari, G., Guberti, V., and Slingenbergh, J. (2011). A review of trends in the distribution of vector-borne diseases: is international trade contributing to their spread? Revue scientifique et technique 30, 119-130.
22. Deredec, A., Burt, A., and Godfray, H. C. (2008). The population genetics of using homing endonuclease genes in vector and pest management. Genetics 179, 2013-2026.
23. Egli, D., Hafen, E., and Schaffner, W. (2004). An efficient method to generate chromosomal rearrangements by targeted DNA double-strand breaks in Drosophila melanogaster. Genome research 14, 1382-1393.
24. Esvelt, K. M., Smidler, A. L., Catteruccia, F., and Church, G. M. (2014). Concerning RNA-guided gene drives for the alteration of wild populations. eLife, e03401.
25. Forster, A., Pannell, R., Drynan, L., Cano, F., Chan, N., Codrington, R., Daser, A., Lobato, N., Metzler, M., Nam, C. H., et al. (2005). Chromosomal translocation engineering to recapitulate primary events of human cancer. Cold Spring Harbor symposia on quantitative biology 70, 275-282.
26. Gantz, V. M. and Bier, E. (2015). The mutagenic chain reaction: A method for converting heterozygous to homozygous mutations. Science.
27. Gibson, D. G., Young, L., Chuang, R. Y., Venter, J. C., Hutchison, C. A., 3rd, and Smith, H. O. (2009). Enzymatic assembly of DNA molecules up to several hundred kilobases. Nature Methods 6, 343-345.
28. Gould, F., and Schliekelman, P. (2004). Population genetics of autocidal control and strain replacement. Annual review of entomology 49, 193-217.
29. Gutierrez, E., Wiggins, D., Fielding, B., and Gould, A. P. (2007). Specialized hepatocyte-like cells regulate Drosophila lipid metabolism. Nature 445, 275-280.
30. Hamilton, W. D. Extraordinary sex ratios. A sex-ratio theory for sex linkage and inbreeding has new implications in cytogenetics and entomology. Science 156, 477-88 (1967).
31. Hay, B. A., Chen, C. H., Ward, C. M., Huang, H., Su, J. T., and Guo, M. (2010). Engineering the genomes of wild insect populations: challenges, and opportunities provided by synthetic Medea selfish genetic elements. Journal of insect physiology 56, 1402-1413.
32. Huang, Y., Magori, K., Lloyd, A. L. & Gould, F. Introducing desirable transgenes into insect populations using Y-linked meiotic drive—a theoretical assessment. Evolution 61, 717-26 (2007).
33. Hurst, G. D. & Werren, J. H. The role of selfish genetic elements in eukaryotic evolution. Nat Rev Genet 2, 597-606 (2001).
34. Jansen, V. A., Turelli, M., and Godfray, H. C. (2008). Stochastic spread of Wolbachia. Proceedings Biological sciences/The Royal Society 275, 2769-2776.
35. Kaiser, P. E., Seawright, J. A., Benedict, M. Q., Narang, S., and Suguna, S. G. (1982). Radiation induced reciprocal translocations and inversions in Anopheles albimanus. Canadian journal of genetics and cytology Journal canadien de genetique et de cytologie 24, 177-188.
36. Kinjo, H., Kunimi, Y. & Nakai, M. Appl Entomol Zool (2014) 49: 297. doi: 10.1007/s13355-014-0249-z
37. Knols, B. G., Bossin, H. C., Mukabana, W. R., and Robinson, A. S. (2007). Transgenic mosquitoes and the fight against malaria: managing technology push in a turbulent GMO world. The American journal of tropical medicine and hygiene 77, 232-242.
38. Krafsur, E. S. (1998). Sterile insect technique for suppressing and eradicating insect populations: 55 years and counting. J Agr Entomol 15, 303-317.
39. Kyrchanova, O., Chetverina, D., Maksimenko, O., Kullyev, A., and Georgiev, P. (2008). Orientation-dependent interaction between Drosophila insulators is a property of this class of regulatory elements. Nucleic acids research 36, 7019-7028.
40. Lambrechts, L., Koella, J. C., and Boete, C. (2008). Can transgenic mosquitoes afford the fitness cost? Trends in parasitology 24, 4-7.
41. Li F, Scott M J (2015) CRISPR/Cas9-mediated mutagenesis of the white and Sex lethal loci in the invasive pest, Drosophila suzukii. Biochem Biophys Res Commun.
42. Magori, K., and Gould, F. (2006). Genetically engineered underdominance for manipulation of pest populations: a deterministic model. Genetics 172, 2613-2620.
43. Mali, P., Esvelt, K. M. & Church, G. M. Cas9 as a versatile tool for engineering biology. Nat Methods 10, 957-63 (2013).
44. Marshall, J. M. (2009). The effect of gene drive on containment of transgenic mosquitoes. Journal of theoretical biology 258, 250-265.
45. Marshall, J. M. (2010). The Cartagena Protocol and genetically modified mosquitoes. Nature biotechnology 28, 896-897.
46. Marshall, J. M., and Hay, B. A. (2012a). Confinement of gene drive systems to local populations: a comparative analysis. Journal of theoretical biology 294, 153-171.
47. Marshall, J. M., and Hay, B. A. (2012b). General principles of single-construct chromosomal gene drive. Evolution; international journal of organic evolution 66, 2150-2166.
48. Marshall, J. M., Pittman, G. W., Buchman, A. B., and Hay, B. A. (2011). Semele: a killer-male, rescue-female system for suppression and replacement of insect disease vector populations. Genetics 187, 535-551.
49. Nicholson, G. M. (2007). Fighting the global pest problem: preface to the special Toxicon issue on insecticidal toxins and their potential for insect pest control. Toxicon: official journal of the International Society on Toxinology 49, 413-422.
50. Oye, K. A., Esvelt, K., Appleton, E., Catteruccia, F., Church, G., Kuiken, T., Lightfoot, S. B., McNamara, J., Smidler, A., and Collins, J. P. (2014). Biotechnology. Regulating gene drives. Science 345, 626-628.
51. Randolph, S. E., and Rogers, D. J. (2010). The arrival, establishment and spread of exotic diseases: patterns and predictions. Nature reviews Microbiology 8, 361-371.
52. Robinson, A. S., and Curtis, C. F. (1973). Controlled Crosses and Cage Experiments with a Translocation in Drosophila. Genetica 44, 591-601.
53. Rong, Y. S., and Golic, K. G. (2003). The homologous chromosome is an effective template for the repair of mitotic DNA double-strand breaks in Drosophila. Genetics 165, 1831-1842.
54. Schmid-Hempel, P. (2005). Evolutionary ecology of insect immune defenses. Annual review of entomology 50, 529-551.
55. Serebrovskii, A. S. On the possibility of a new method for the control of insect pests. Zoologicheskii Zhurnal 19, 618-630 (1940).
56. Schetelig M F, Handler A M (2013) Germline transformation of the spotted wing drosophilid, Drosophila suzukii, with a piggyBac transposon vector. Genetica 141:189-193.
57. Sherizen, D., Jang, J. K., Bhagat, R., Kato, N., and McKim, K. S. (2005). Meiotic recombination in Drosophila females depends on chromosome continuity between genetically defined boundaries. Genetics 169, 767-781.
58. Sinha, S., Medhi, B. & Sehgal, R. Challenges of drug-resistant malaria. Parasite 21, 61 (2014).
59. Sinkins, S. P., and Gould, F. (2006). Gene drive systems for insect disease vectors. Nature reviews Genetics 7, 427-435.
60. Tatem, A. J., Rogers, D. J., and Hay, S. I. (2006). Global transport networks and infectious disease spread. Advances in parasitology 62, 293-343.
61. Theilmann, D. A., and Stewart, S. (1992). Molecular analysis of the trans-activating IE-2 gene of Orgyia pseudotsugata multicapsid nuclear polyhedrosis virus. Virology 187, 84-96.
62. Tripet, F., Aboagye-Antwi, F., and Hurd, H. (2008). Ecological immunology of mosquito-malaria interactions. Trends in parasitology 24, 219-227.
63. Uemura, M., Niwa, Y., Kakazu, N., Adachi, N., and Kinoshita, K. (2010). Chromosomal manipulation by site-specific recombinases and fluorescent protein-based vectors. PloS one 5, e9846.
64. Werren, J. H. Selfish genetic elements, genetic conflict, and evolutionary innovation. Proc Natl Acad Sci USA 108 Suppl 2, 10863-70 (2011).
65. Willis, N. L., Seawright, J. A., Nickel, C., and Joslyn, D. J. (1981). Reciprocal translocations and partial correlation of chromosomes in the stable fly. The Journal of heredity 72, 104-106.
66. Windbichler, N., Menichelli, M., Papathanos, P. A., Thyme, S. B., Li, H., Ulge, U. Y., Hovde, B. T., Baker, D., Monnat, R. J., Jr., Burt, A., et al. (2011). A synthetic homing endonuclease-based gene drive system in the human malaria mosquito. Nature 473, 212-215.
67. Yu, Y., and Bradley, A. (2001). Engineering chromosomal rearrangements in mice. Nature reviews Genetics 2, 780-790.
68. Bassett, A. R., Tibbit, C., Ponting, C. P. & Liu, J. L. Highly efficient targeted mutagenesis of Drosophila with the CRISPR/Cas9 system. Cell Rep 4, 220-8 (2013).
69. Basu, S. et al. Silencing of end-joining repair for efficient site-specific gene insertion after TALEN/CRISPR mutagenesis in Aedes aegypti. Proc Natl Acad Sci USA 112, 4038-43 (2015).
70. Yu, Z. et al. Highly efficient genome modifications mediated by CRISPR/Cas9 in Drosophila. Genetics 195, 289-91 (2013).
71. O'Connell, M. R. et al. Programmable RNA recognition and cleavage by CRISPR/Cas9. Nature 516, 263-6 (2014).
72. Price, A. A., Sampson, T. R., Ratner, H. K., Grakoui, A. & Weiss, D. S. Cas9-mediated targeting of viral RNA in eukaryotic cells. Proc Natl Acad Sci USA 112, 6164-9 (2015).
73. Gao, Y. & Zhao, Y. Self-processing of ribozyme-flanked RNAs into guide RNAs in vitro and in vivo for CRISPR-mediated genome editing. J Integr Plant Biol 56, 343-9 (2014).
74. Xie, K., Minkenberg, B. & Yang, Y. Boosting CRISPR/Cas9 multiplex editing capability with the endogenous tRNA-processing system. Proc Natl Acad Sci USA 112, 3570-5 (2015).
75. Xie, S., Shen, B., Zhang, C., Huang, X. & Zhang, Y. sgRNAcas9: a software package for designing CRISPR sgRNA and evaluating potential off-target cleavage sites. PLoS One 9, e100448 (2014).
76. Marshall, J. M. A., O. S. in Genetic Control of Malaria and Dengue (ed. Adelman, Z. N.) (2015).
77. Bull, J. J. Evolutionary decay and the prospects for long-term disease intervention using engineered insect vectors. Evol Med Public Health 2015, 152-66 (2015).
78. Stoddard, B. L. Homing endonucleases: from microbial genetic invaders to reagents for targeted DNA modification. Structure 19, 7-15 (2011).
79. Windbichler, N. et al. A synthetic homing endonuclease-based gene drive system in the human malaria mosquito. Nature 473, 212-5 (2011).
80. Windbichler, N. et al. Homing endonuclease mediated gene targeting in Anopheles gambiae cells and embryos. Nucleic Acids Res 35, 5922-33 (2007).
81. Chan, Y. S., Huen, D. S., Glauert, R., Whiteway, E. & Russell, S. Optimising homing endonuclease gene drive performance in a semi-refractory species: the Drosophila melanogaster experience. PLoS One 8, e54130 (2013).
82. Chan, Y. S., Naujoks, D. A., Huen, D. S. & Russell, S. Insect population control by homing endonuclease-based gene drive: an evaluation in Drosophila melanogaster. Genetics 188, 33-44 (2011).
83. Chan, Y. S. et al. The design and in vivo evaluation of engineered I-Onul-based enzymes for HEG gene drive. PLoS One 8, e74254 (2013).
84. Simoni, A. et al. Development of synthetic selfish elements based on modular nucleases in Drosophila melanogaster. Nucleic Acids Res 42, 7461-72 (2014).
85. Tsai, S. Q. et al. GUIDE-seq enables genome-wide profiling of off-target cleavage by CRISPR-Cas nucleases. Nat Biotechnol 33, 187-97 (2015).
86. Lyttle, T. W. Cheaters sometimes prosper: distortion of mendelian segregation by meiotic drive. Trends Genet 9, 205-10 (1993).
87. Wood, R. J. & Newton, M. E. Sex-Ratio Distortion Caused by Meiotic Drive in Mosquitoes. The American Naturalist 137, 379-391 (1991).
88. Papathanos, P. A. W., N.; Akbari, O. S. in Transgenic insects: techniques and applications (ed. Benedict, M. Q.) (2014).
89. Helleu, Q., Gerard, P. R. & Montchamp-Moreau, C. Sex chromosome drive. Cold Spring Hart, Perspect Biol 7, a017616 (2015).
90. Craig, G. B., Jr., Hickey, W. A. & Vandehey, R. C. An inherited male-producing factor in Aedes aegypti. Science 132, 1887-9 (1960).
91. Hickey, W. A. & Craig, G. B., Jr. Distortion of sex ratio in populations of Aedes aegypti. Can J Genet Cytol 8, 260-78 (1966).
92. Lyttle, T. W. Experimental population genetics of meiotic drive systems. I. Pseudo-Y chromosomal drive as a means of eliminating cage populations of Drosophila melanogaster. Genetics 86, 413-45 (1977).
93. Newton, M. E., Wood, R. J. & Southern, D. I. A cytogenetic analysis of meiotic drive in the mosquito, Aedes aegypti (L.). Genetica 46, 297-318 (1976).
94. Deredec, A., Burt, A. & Godfray, H. C. The population genetics of using homing endonuclease genes in vector and pest management. Genetics 179, 2013-26 (2008).
95. Klein, T. A., Windbichler, N., Deredec, A., Burt, A. & Benedict, M. Q. Infertility resulting from transgenic I-PpoI male Anopheles gambiae in large cage trials. Pathog Glob Health 106, 20-31 (2012).
96. Windbichler, N., Papathanos, P. A. & Crisanti, A. Targeting the X chromosome during spermatogenesis induces Y chromosome transmission ratio distortion and early dominant embryo lethality in Anopheles gambiae. PLoS Genet 4, e1000291 (2008).
97. Galizi, R. et al. A synthetic sex ratio distortion system for the control of the human malaria mosquito. Nat Commun 5, 3977 (2014).
98. Bernardini, F. et al. Site-specific genetic engineering of the Anopheles gambiae Y chromosome. Proc Natl Acad Sci USA 111, 7600-5 (2014).
99. Wade, M. J. & Beeman, R. W. The population dynamics of maternal-effect selfish genes. Genetics 138, 1309-14 (1994).
100. Foster, G. G., Whitten, M. J., Prout, T. & Gill, R. Chromosome rearrangements for the control of insect pests. Science 176, 875-80 (1972).
101. Coghlan, A., Eichler, E. E., Oliver, S. G., Paterson, A. H. & Stein, L. Chromosome evolution in eukaryotes: a multi-kingdom perspective. Trends Genet 21, 673-82 (2005).
102. Asman, S. M., McDonald, P. T. & Prout, T. Field studies of genetic control systems for mosquitoes. Annu Rev Entomol 26, 289-318 (1981).
103. Baker, R. H. Chromosome rearrangements in the control of mosquitoes. Preventive Veterinary Medicine 2, 529-540 (1984).
104. Laven, H., Cousserans, J. & Guille, G. Eradicating mosquitoes using translocations: a first field experiment. Nature 236, 456-7 (1972).
105. Robinson, A. S. Progress in the use of chromosomal translocations for the control of insect pests. Biol Rev Camb Philos Soc 51, 1-24 (1976).
106. Reeves, R. G., Bryk, J., Altrock, P. M., Denton, J. A. & Reed, F. A. First steps towards underdominant genetic transformation of insect populations. PLoS One 9, e97557 (2014).
107. Sherizen, D., Jang, J. K., Bhagat, R., Kato, N. & McKim, K. S. Meiotic recombination in Drosophila females depends on chromosome continuity between genetically defined boundaries. Genetics 169, 767-81 (2005).
108. Serbus, L. R., Casper-Lindley, C., Landmann, F. & Sullivan, W. The genetics and cell biology of Wolbachia-host interactions. Annu Rev Genet 42, 683-707 (2008).
109. Hertig, M. & Wolbach, S. B. Studies on Rickettsia-Like Micro-Organisms in Insects. J Med Res 44, 329-374 7 (1924).
110. Moreira, L. A. et al. A Wolbachia symbiont in Aedes aegypti limits infection with dengue, Chikungunya, and Plasmodium. Cell 139, 1268-78 (2009).
111. Bian, G. et al. Wolbachia invades Anopheles stephensi populations and induces refractoriness to Plasmodium infection. Science 340, 748-51 (2013).
112. Bian, G., Zhou, G., Lu, P. & Xi, Z. Replacing a native Wolbachia with a novel strain results in an increase in endosymbiont load and resistance to dengue virus in a mosquito vector. PLoS Negl Trop Dis 7, e2250 (2013).
113. Blagrove, M. S., Arias-Goeta, C., Failloux, A. B. & Sinkins, S. P. Wolbachia strain wMel induces cytoplasmic incompatibility and blocks dengue transmission in Aedes albopictus. Proc Natl Acad Sci USA 109, 255-60 (2012).
114. Kambris, Z., Cook, P. E., Phuc, H. K. & Sinkins, S. P. Immune activation by life-shortening Wolbachia and reduced filarial competence in mosquitoes. Science 326, 134-6 (2009).
115. Sinkins, S. P. Wolbachia and arbovirus inhibition in mosquitoes. Future Microbiol 8, 1249-56 (2013).
116. McMeniman, C. J. et al. Stable introduction of a life-shortening Wolbachia infection into the mosquito Aedes aegypti. Science 323, 141-4 (2009).
117. Rasgon, J. L., Styer, L. M. & Scott, T. W. Wolbachia-induced mortality as a mechanism to modulate pathogen transmission by vector arthropods. J Med Entomol 40, 125-32 (2003).
118. Hoffmann, A. A. et al. Successful establishment of Wolbachia in Aedes populations to suppress dengue transmission. Nature 476, 454-7 (2011).
119. Walker, T. et al. The wMel Wolbachia strain blocks dengue and invades caged Aedes aegypti populations. Nature 476, 450-3 (2011).
120. Rasgon, J. L. & Gould, F. Transposable element insertion location bias and the dynamics of gene drive in mosquito populations. Insect Mol Biol 14, 493-500 (2005).
121. Smith, R. C. & Atkinson, P. W. Mobility properties of the Hermes transposable element in transgenic lines of Aedes aegypti. Genetica 139, 7-22 (2011).
122. Houben, A., Banaei-Moghaddam, A. M., Klemme, S. & Timmis, J. N. Evolution and biology of supernumerary B chromosomes. Cell Mol Life Sci 71, 467-78 (2014).
123. Akbari, O. S., Antoshechkin, I., Hay, B. A. & Ferree, P. M. Transcriptome profiling of Nasonia vitripennis testis reveals novel transcripts expressed from the selfish B chromosome, paternal sex ratio. G3 (Bethesda) 3, 1597-605 (2013).
124. Gould, F., Huang, Y., Legros, M. & Lloyd, A. L. A killer-rescue system for self-limiting gene drive of anti-pathogen constructs. Proc Biol Sci 275, 2823-9 (2008).
125. Marshall, J. M. & Hay, B. A. Inverse Medea as a novel gene drive system for local population replacement: a theoretical analysis. J Hered 102, 336-41 (2011).
126. Marshall, J. M. The toxin and antidote puzzle: new ways to control insect pest populations through manipulating inheritance. Bioeng Bugs 2, 235-40 (2011).
127. Marshall, J. M. & Hay, B. A. General principles of single-construct chromosomal gene drive. Evolution 66, 2150-66 (2012).
128. Marshall, J. M. & Hay, B. A. Medusa: a novel gene drive system for confined suppression of insect populations. PLoS One 9, e102694 (2014).
129. Oye, K. A. et al. Biotechnology. Regulating gene drives. Science 345, 626-8 (2014).
130. Knols, B. G., Bossin, H. C., Mukabana, W. R. & Robinson, A. S. Transgenic mosquitoes and the fight against malaria: managing technology push in a turbulent GMO world. Am J Trop Med Hyg 77, 232-42 (2007).
131. Gurwitz, D. Gene drives raise dual-use concerns. Science 345, 1010 (2014).
132. Oye, K. A. & Esvelt, K. M. Gene drives raise dual-use concerns—response. Science 345, 1010-1 (2014).
133. McNaughton, D. The importance of long-term social research in enabling participation and developing engagement strategies for new dengue control technologies. PLoS Negl Trop Dis 6, e1785 (2012).
134. Marshall, J. M., Toure, M. B., Traore, M. M., Famenini, S. & Taylor, C. E. Perspectives of people in Mali toward genetically-modified mosquitoes for malaria control. Malar J 9, 128 (2010).
135. Alphey, L., Nimmo, D., O'Connell, S. & Alphey, N. Insect population suppression using engineered insects. Adv Exp Med Biol 627, 93-103 (2008).
136. Carvalho, D. O. et al. Suppression of a Field Population of Aedes aegypti in Brazil by Sustained Release of Transgenic Male Mosquitoes. PLoS Negl Trop Dis 9, e0003864 (2015).
137. Harris, A. F. et al. Successful suppression of a field mosquito population by sustained release of engineered male mosquitoes. Nat Biotechnol 30, 828-30 (2012).
138. Harris, A. F. et al. Field performance of engineered male mosquitoes. Nat Biotechnol 29, 1034-7 (2011).
139. Lacroix, R. et al. Open field release of genetically engineered sterile male Aedes aegypti in Malaysia. PLoS One 7, e42771 (2012).
140. Akbari, O. S. et al. Safeguarding gene drive experiments in the laboratory. Science 349, 927-9 (2015).
141. James, A. A. Gene drive systems in mosquitoes: rules of the road. Trends Parasitol 21, 64-7 (2005).
142. David, A. S., Kaser, J. M., Morey, A. C., Roth, A. M. & Andow, D. A. Release of genetically engineered insects: a framework to identify potential ecological effects. Ecol Evol 3, 4000-15 (2013).
143. Wolbers, M., Kleinschmidt, I., Simmons, C. P. & Donnelly, C. A. Considerations in the design of clinical trials to test novel entomological approaches to dengue control. PLoS Negl Trop Dis 6, e1937 (2012).
144. Marshall, J. M. The effect of gene drive on containment of transgenic mosquitoes. J Theor Biol 258, 250-65 (2009).
145. Marshall, J. M. The Cartagena Protocol and genetically modified mosquitoes. Nat Biotechnol 28, 896-7 (2010).
146. Walsh, Douglas B., Mark P. Bolda, Rachael E. Goodhue, Amy J. Dreves, Jana Lee, Denny J. Bruck, Vaughn M. Walton, Sally D. O'Neal, and Frank G. Zalom. “Drosophila suzukii (Diptera: Drosophilidae): invasive pest of ripening soft fruit expanding its geographic range and damage potential.” Journal of Integrated Pest Management 2, no. 1 (2011): G1-G7.
147. Asplen, Mark K., Gianfranco Anfora, Antonio Biondi, Deuk-Soo Choi, Dong Chu, Kent M. Daane, Patricia Gibert et al. “Invasion biology of spotted wing Drosophila (Drosophila suzukii): a global perspective and future priorities.” Journal of Pest Science 88, no. 3 (2015): 469-494.
148. Gabarra, Rosa, Jordi Riudavets, Gustavo A. Rodriguez, Juli Pujade-Villar, and Judit Arn6. “Prospects for the biological control of Drosophila suzukii.” BioControl 60, no. 3 (2014): 331-339.
149. Woltz, J. M., K. M. Donahue, D. J. Bruck, and J. C. Lee. “Efficacy of commercially available predators, nematodes and fungal entomopathogens for augmentative control of Drosophila suzukii.” Journal of Applied Entomology (2015).
150. Clark, Melody S., and M. Roger Worland. “How insects survive the cold: molecular mechanisms—a review.” Journal of Comparative Physiology B 178, no. 8 (2008): 917-933.
151. Schetelig, Marc F., and Alfred M. Handler. “Germline transformation of the spotted wing drosophilid, Drosophila suzukii, with a piggyBac transposon vector.” Genetica 141, no. 4-6 (2013): 189-193.
152. Kambris, Zakaria, Hana Bilak, Rosalba D′Alessandro, Marcia Belvin, Jean-Luc Imler, and Maria Capovilla. “DmMyD88 controls dorsoventral patterning of the Drosophila embryo.” EMBO reports 4, no. 1 (2003): 64-69.
153. Schejter, Eyal D., and Eric Wieschaus. “bottleneck acts as a regulator of the microfilament network governing cellularization of the Drosophila embryo.” Cell 75, no. 2 (1993): 373-385.
154. Berghammer, Andreas J., Martin Klingler, and Ernst A. Wimmer. “Genetic techniques: a universal marker for transgenic insects.” Nature 402, no. 6760 (1999): 370-371.
155. Theilmann, David A., and Sandra Stewart. “Tandemly repeated sequence at the 3′ end of the IE-2 gene of the baculovirus Orgyia pseudotsugata multicapsid nuclear polyhedrosis virus is an enhancer element.” Virology 187, no. 1 (1992): 97-106.
156. Dyck, V. Arnold, Jorge Hendrichs, and Alan S. Robinson. Sterile insect technique. IAEA, 2005.
157. Gitzinger, Marc, Christian Kemmer, David A. Fluri, Marie Daoud El-Baba, Wilfried Weber, and Martin Fussenegger. “The food additive vanillic acid controls transgene expression in mammalian cells and mice.” Nucleic acids research (2011): gkr1251.
158. Urlinger, Stefanie, Udo Baron, Marion Thellmann, Mazahir T. Hasan, Hermann Bujard, and Wolfgang Hillen. “Exploring the sequence space for tetracycline-dependent transcriptional activators: novel mutations yield expanded range and sensitivity.” Proceedings of the National Academy of Sciences 97, no. 14 (2000): 7963-7968.
159. Fu, Guoliang, Kirsty C. Condon, Matthew J. Epton, Peng Gong, Li Jin, George C. Condon, Neil I. Morrison, Tarig H. Dafa'alla, and Luke Alphey. “Female-specific insect lethality engineered using alternative splicing.” Nature biotechnology 25, no. 3 (2007): 353-357.
160. Bachmann and Kust, The Use of P-Element Transposons to Generate Transgenic Flies (Chapter 4), Methods in Molecular Biology: Drosophila: Methods and Protocols Edited by: C. Dahmann C Humana Press Inc., Totowa, N. J.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

Use of the singular forms “a,” “an,” and “the” include plural references unless the context clearly dictates otherwise.

Unless defined otherwise or the context clearly dictates otherwise, all technical and scientific terms used herein have the same meaning as commonly understood by one of ordinary skill in the art to which this invention belongs.

1. A gene drive system for biocontrol of a Drosophila suzukii population, comprising
a first DNA sequence encoding a toxin under the control of a maternal germline-specific promoter active in D. suzukii, and linked to
a second DNA sequence encoding an antidote under the control of an early embryo-specific promoter active in D. suzukii,
wherein the toxin is expressed in D. suzukii maternal germline cells and results in maternal-effect lethality in D. suzukii, and the antidote is expressed in D. suzukii embryos and counters the maternal-effect lethality.

2. The gene drive system of claim 1, wherein the toxin comprises one or more miRNAs or RNA-guided endonucleases, or a combination thereof.

3. The gene drive system of claim 1, wherein the toxin targets a gene for a D. suzukii maternally-deposited embryonic-essential RNA or protein.

4. The gene drive system of claim 3, wherein the target gene is myd88.

5. The gene drive system of claim 3, wherein the antidote is a toxin-resistant version of the target gene.

6. The gene drive system of claim 5, wherein the toxin-resistant version is not recognized by the toxin.

7. The gene drive system of claim 1, wherein the maternal germline-specific promoter is a bicoid promoter.

8. The gene drive system of claim 1, wherein the early embryo-specific promoter is a bottleneck promoter

9. The gene drive system of claim 1, further comprising an effector genetic element active in D. suzukii and linked to the first and second DNA sequences, wherein the effector genetic element encodes a gene conferring susceptability to a chemical, a conditional lethal gene, a genetic element that disrupts a recessive fertility gene or recessive lethality gene, or a genetic element that disrupts a gene involved in D. suzukii pest behavior.

10. Transgenic D. suzukii having a genome comprising the gene drive system of claim 1.

11. A method of manipulating a D. suzukii population, the method comprising releasing a transgenic D. suzukii having a genome comprising the gene drive system of claim 9 into the population in sufficient numbers to spread the gene drive system through the population.

12. The method of claim 11, wherein the effector genetic element results in inducible lethality in one or both sexes of D. suzukii or results in recessive sterility in one or both sexes of D. suzukii.

13. A method of manipulating a D. suzukii population, the method comprising releasing the transgenic D. suzukii of claim 10 into the population in sufficient numbers to spread the gene drive system through the population.

14. A gene drive system for biocontrol of a population of an insect, comprising
a first DNA sequence encoding a toxin under the control of a maternal germline-specific promoter active in the insect, and linked to
a second DNA sequence encoding an antidote under the control of an early embryo-specific promoter active in the insect,
wherein the toxin is expressed in maternal germline cells of the insect and results in maternal-effect lethality in the insect, and the antidote is expressed in embryos of the insect and counters the maternal-effect lethality, and
wherein the insect is not Drosophila melanogaster or a flour beetle.

15. The gene drive system of claim 14, wherein the toxin is an miRNA or an endonuclease.

16. The gene drive system of claim 14, wherein the toxin targets a gene for a maternally-deposited embryonic-essential RNA or protein of the insect.

17. The gene drive system of claim 16, wherein the target gene is myd88.

18. The gene drive system of claim 16, wherein the antidote is a toxin-resistant version of the target gene.

19. The gene drive system of claim 18, wherein the toxin-resistant version is not recognized by the toxin.

20. The gene drive system of claim 14, further comprising an effector genetic element active in the insect and linked to the first and second DNA sequences, wherein the effector genetic element encodes a gene conferring susceptibility to a chemical, a conditional lethal gene, a genetic element that disrupts a recessive fertility gene or recessive lethality gene, or a genetic element that disrupts a gene involved in insect pest behavior.

21. The gene drive system of claim 14, wherein the insect is Drosophila suzukii, Anastrepha suspensa, Anastrepha ludens, Anastrepha oblique, Bactrocera oleae/Dacus oleae, Ceratitis capitata, Aedes aegyptii, or Anopheles gambiae.

22. A transgenic insect whose genome comprises the gene drive system of claim 14.

23. A method of manipulating an insect population, the method comprising releasing the transgenic insect of claim 22 into a population of the same species in sufficient numbers to spread the gene drive system through the population.

24. A method of manipulating an insect population, the method comprising releasing a transgenic insect having a gene drive system of claim 20 into a population of the same species in sufficient numbers to spread the gene drive system through the population, wherein the effector genetic element results in inducible lethality in one or both sexes of the species or results in recessive sterility in one or both sexes of the species.
